(19) United States
(12) Patent Application Publication
(10) Pub. No.: US 2022/0071234 A1
(43) Pub. Date: Mar. 10, 2022

(54) PEA-BASED DRY PRODUCT FOR FEEDING ANIMALS

(71) Applicant: ROQUETTE FRERES, Lestrem (FR)

(72) Inventors: Christian DELPORTE, Anhiers (FR); Samuel PATINIER, Quesnoy Sur Deule (FR)

(21) Appl. No.: 17/309,797

(22) PCT Filed: Dec. 20, 2019
(86) PCT No.: PCT/FR2019/053251

§ 371 Date: Jun. 18, 2021

(30) Foreign Application Priority Data
Dec. 21, 2018 (FR) 1873878

(51) Int. Cl. A23K 10/30; A23K 30/20; A23K 40/00; A23K 50/30; A23L 11/00; A61K 8/9789; A61K 47/46

(52) U.S. Cl. CPC A23K 10/30; A23K 30/20; A23K 40/00; A23V 2002/00; A23L 11/05; A61K 8/9789; A61K 47/46; A23K 50/30

(57) ABSTRACT

The invention relates to a dry product based on peas, to the process for preparing same and to the use thereof.

Plant biorefinery is a fast-growing industrial activity. It aims to extract and to upgrade the various compounds present in plants, in order to profitably use them in various industrial sectors ranging from food for human consumption to cosmetics, and also the pharmaceutical industry.

Several classifications exist and one of them consists in sorting the sectors according to the botanical source used as the raw source of compounds, e.g. the wheat sector or the corn sector.

In order to sustain its economic model, each sector must make use of all of the extracted products. It is thus standard practice to extract and isolate, mainly via wet processes, the various constituents of these plants into concentrated fractions such as starch or proteins.

This extraction by depletion also results in the subsequent creation of fractions usually called “by-products”, which, although they do not consist of pure and concentrated fractions, for instance starch, they must nevertheless be the subject of economic exploitation. These by-products are also called “secondary products”.

This is the case, for example, for the wheat and corn sectors, which, after the extraction of starch and proteins, mainly generate liquid by-products, usually called “soluble fractions”, which mainly contain soluble proteins, which are difficult to extract, and also water-soluble salts and sugars. Another type of common by-product is well known under the term “pulps” or “fibres”, which term encompasses the fractions predominantly containing fibres which are resistant to human digestion.

Within these sectors, by-products are usually mixed and then dried so that they can be used as nutrients in animal feed, by virtue of their high content of fibres and protein. For instance, stillage (Distillers Dried Grains with Solubles “DDGS”) or starch sector by-products (“Corn Gluten Feed” or “Wheat Gluten Feed”) are well known in the field.

These mixed and dried by-products are very valuable for the animal nutrition industry; however, their production has some residual drawbacks.

Specifically, the drying of the mixture of these two by-products, which produces a mixture of soluble proteins and of sugars (from fibres or residues) is troublesome due to potential Maillard reactions, which results in some colouring.

The appearance of this colouring is linked to the denaturing of the amino acids, particularly of lysine, during Maillard reactions involving the reducing sugars present in the product, and which take place during drying. The correlation of the colour measured by its L*a*b* chromatic space, and in particular its component L, with the lysine content, is well known in these fields, particularly in DDGSs (see, for example, the presentation “The Effects of Drying on DDGS Protein Quality” from the company GEA).

The Maillard reactions also have the consequence of degrading the digestibility of the product obtained after drying. This degradation is amply described in the publications “US GRAINS COUNCIL—A guide to Distiller's Dried Grains with Solubles—Third Edition” and “Journées de la Recherche Porcine 2009—Valeur nutritionnelle des drêches de blé européennes chez le porc en croissance [Conference on Porcine Research 2009—Nutritional value of European wheat stillage grain for growing pigs]”.

According to these publications, the digestibility of the organic matter may fall to between 60% and 45% in pigs, when the co-drying generates browning reactions.

The pea sector also produces pulp fractions and soluble fractions. These are of good nutritional value for animals. This nutritional value was studied at the pig research station of the INRA, Rennes (France), during two series of measurements of in vivo digestibility in growing pigs.

In 2005, the INRA measured the total digestibility of the constituents of wet pea pulp and of liquid pea soluble matter, in comparison with that of ground raw peas. In 2006, the study focused on the ileal digestibility of protein and of amino acids. These two studies were published at the 39th Conference on Porcine Research—Paris 2007: “Nutritional value in pigs of by-products from the pea starch sector” (Jean Noblet, Yolande Jaguelin-Peyraud, Bernard Sève, Christian Delporte).

The total digestibility of the organic matter in pigs, which are monogastric animals, was measured, according to the INRA protocol, to be 91.8% for pea pulp, and 93.8% for pea soluble matter, in comparison with a value of 90.7% for raw peas. The ileal digestibility of lysine was 82.2% for pea pulp, and 90.8% for pea soluble matter, as opposed to 84.9% for raw peas.

It is important to note that these two by-products were studied in their wet form. Specifically, the leguminous plant sectors, in particular the pea sector, have completely set aside co-drying of these by-products, particularly the internal fibres and the fraction called “soluble fraction”. Specifically, peas have, in the residual proteins present in the soluble fraction, a lysine content which is higher than in the case of corn or wheat. Those skilled in the art, well aware of the Maillard reactions, have thus been strongly discouraged from drying the mixture of these two by-products. In the document “Sector reference document on the manufacturing of safe feed materials from starch processing. Version 3” published in June 2014 by the European Guide to good practice for the industrial manufacture of safe feed materials, it may be seen that mixing followed by drying of coproducts is clearly indicated in corn and wheat sector processes (cf. “Maize Gluten Feed” page 11 and “Wheat Gluten Feed” page 13). It can also be seen on page 18 that the mixture of dried by-products is totally disregarded in the pea sector.

It is thus advantageous for the pea sector to obtain a dry product produced by mixing its by-products, in particular pulps and soluble fractions, followed by drying, in order to upgrade as best as possible the sector as a whole, by optimization of its nutritional profile and by simplification of the upgrading of these by-products.

It is to the Applicant's credit to have worked on this issue and to have designed the invention, the description of which is given in the following chapter.

### DESCRIPTION OF THE INVENTION

A first subject of the invention relates to the product derived from the mixing of a soluble fraction from leguminous plants and of pulps from leguminous plants, the solids content of which is greater than 85% by weight, preferentially greater than 90% by weight, even more preferentially greater than 94% by weight.

The leguminous plant is preferentially chosen from the list composed of faba bean and pea. Pea is particularly preferred.

Preferably, the product in the form of a powder is characterized in that 80%, preferentially 90%, of its particle size distribution has a size of between 50 microns and 3000 microns, preferentially between 100 microns and 2000 microns, even more preferentially between 300 microns and 1000 microns.

Preferentially, the product according to the invention has an “L*a*b*” colouring characterized in that its component L is greater than 30, preferentially greater than 40, even more preferentially greater than 50.

More preferentially, the L*a*b* colouring of the product according to the invention is also characterized by its component a of less than 20, preferentially less than 10, and its component b of greater than 25, preferably greater than 30, preferably greater than 40, preferentially greater than 50.

Even more preferentially, the product according to the invention has a lysine content of between 3% and 10% by weight based on its total protein content, preferentially between 5% and 8% by weight.

Finally, even more preferentially, the product according to the invention is characterized in that the total digestibility of its organic matter for monogastric animals is greater than 75%.

The invention also relates to a process for preparing the product derived from the mixing of a soluble fraction from leguminous plants and of pulps from leguminous plants, the solids content of which is greater than 85% by weight, preferentially greater than 90% by weight, even more preferentially greater than 94% by weight, comprising the following steps:

i) pretreatment of leguminous plant seeds;
ii) wet separation of the constituents of the leguminous plant seeds into four fractions: a starch fraction, a pulp fraction, a protein fraction of globulin type and a soluble fraction;
iii) mixing of the pulp fraction and the soluble fraction separated in the preceding step ii);
iv) drying of the mixture obtained in step iii).

Preferentially, the leguminous plant is chosen from the list composed of faba bean and pea. Pea is particularly preferred.

Preferably, the ratio, expressed in solids content, of the soluble fraction to the pulp fraction is between 0.8/1.2 and 1.2/0.8, preferentially of 1/1.

Preferably, the soluble fraction is pre-concentrated to between 30% and 50% by weight, preferentially to an S.C. (solids content) of 50% by weight before being mixed with the fibres, also known as the pulp fraction.

Even more preferably, the mixing of the pulp fraction and of the soluble fraction is performed in a high-performance mixer for a residence time of less than 5 min.

Preferably, drying is performed via a ring-dryer technology. Preferably, the evaporation mists obtained during drying are recycled.

Finally, the invention also relates to the industrial use of the mixture of a soluble fraction from leguminous plants and of pulps from leguminous plants, the solids content of which is greater than 85% by weight, preferentially greater than 90% by weight, even more preferentially greater than 94% by weight, in industrial applications such as food for human consumption, animal feed, pharmaceuticals or cosmetics.

The invention will be better understood with the aid of the following detailed description.

### DETAILED DESCRIPTION OF THE INVENTION

A first subject of the invention relates to the product derived from the mixing of a soluble fraction from leguminous plants and of pulps from leguminous plants, the solids content of which is greater than 85% by weight, preferentially greater than 90% by weight, even more preferentially greater than 94% by weight.

Preferably, the leguminous plant is preferentially chosen from the list composed of faba bean and pea. Pea is particularly preferred.

The solids content is measured by any method that is well known to those skilled in the art. Preferentially, the “desiccation” method is used. It consists in determining the amount of water evaporated by heating a known amount of a sample of known mass:
The sample is first weighed and a mass m1 is measured in grams.
The water is evaporated off by placing the sample in a heated chamber until the mass of the sample has stabilized, the water being completely evaporated off. Preferably, the temperature is 105° C. at atmospheric pressure.
The final sample is weighed and a mass m2 is measured in grams.

Solids content=(m2/m1)×100

The term “soluble fraction from leguminous plants” means the residual aqueous fraction after the extraction of the starch, pulps and proteins of globulin type derived from seeds of leguminous plants, using a “wet” fractionation process. Such a process is, for example, the process described by the Applicant in patent application EP1400537, which is incorporated herein by reference. The principle of this process consists in grinding the leguminous plant seed to obtain a meal, which is then resuspended in water. Using decanters and hydrocyclones, the starch and the internal fibres (pulps) are separated. The residual solution, having a high protein content, is acidified to a pH of about 4.5, and then undergoes a heating step to coagulate the proteins of globulin type, which will be separated by centrifugation. The residual solution consists of the “soluble fraction”. This process makes it possible to obtain pea soluble fractions and pea pulps (cf. paragraphs 105 and 106). It can be modified by adding, for example, a step of soaking, of toasting (heating of the grains to dryness). This soluble fraction of a leguminous plant consists primarily of proteins which are soluble at an acidic pH, belonging mainly to the group of albumins, and also various water-soluble compounds such as sugars and salts. The soluble fraction of a leguminous plant may also undergo a heat treatment, which makes it possible to remove anti-nutritional factors such as trypsin inhibitors.

The term “pulps” or “fibres” means all of the non-starchy polysaccharides which can be extracted by centrifugation as part of a “wet” fractionation process, as described in the preceding paragraph. Reference may be made to the reference document “Sector reference document on the manufacturing of safe feed materials from starch processing. Version 3” published in June 2014 by the European Guide to good practice for the industrial manufacture of safe feed materials. The term “pulps” or “fibres” will particularly mean the fraction known as “pea pulp”.

Thee term “leguminous plants” is considered here to be the family of dicot plants of the order of Fabales. It is one of the most important families of flowering plants, the third ranking after Orchidaceae and Asteraceae regarding the number of species. It contains about 765 genera combining more than 19 500 species. Several leguminous plants are significant crop plants, such as peas, faba bean, lupin, beans, chickpea, groundnut, cultivated lentil, broad beans, locust bean, liquorice.

The term “pea” is considered here in its broadest sense and includes in particular all wild-type varieties of “smooth pea” and “wrinkled pea” and all mutant varieties of “smooth pea” and “wrinkled pea”, irrespective of the uses for which said varieties are generally intended (food for human consumption, animal feed and/or other uses).

In the present patent application, the term “pea” includes the varieties of pea belonging to the Pisum genus and more particularly to the sativum and aestivum species. Said mutant varieties are notably those known as “r mutants”, “rb mutants”, “rug 3 mutants”, “rug 4 mutants”, “rug 5 mutants” and “lam mutants” as described in the article by C-L Heydley et al., entitled “Developing novel pea starches”, Proceedings of the Symposium of the Industrial Biochemistry and Biotechnology Group of the Biochemical Society, 1996, pages 77-87.

Preferably, the particle size of the product in the form of a powder is characterized in that 80%, preferentially 90% of its particle size distribution has a size of between 50 microns and 3000 microns, preferentially between 100 microns and 2000 microns, even more preferentially between 300 microns and 1000 microns.

The particle size will be measured by any technique that is well known to those skilled in the art. Laser particle size analysis will preferably be used. This particle size analysis will be expressed as a volume distribution.

Preferentially, the product according to the invention has an “L*a*b*” colouring characterized in that its component L is greater than 30, preferentially greater than 40.

Alternatively, the L*a*b* colouring of the product according to the invention is also characterized by its component a of less than 20, preferentially less than 10, and its component b of greater than 25, preferably greater than 30, preferably greater than 40, preferentially greater than 50.

The term “L*a*b* colouring” means the evaluation of the colouring, using a suitable spectrophotometer, which converts it into three parameters:
the lightness L having values from 0 (black) to 100 (reference white);
the parameter a representing the value on the green→red axis;
the parameter b representing the value on the blue→yellow axis.

The measurement of this colouring is preferentially performed using the spectrophotometers DATA COLOR—DATA FLASH 100 or KONIKA MINOLTA CMS, with the aid of their user manuals.

Even more preferentially, the product according to the invention has a lysine content of between 3% and 10% by weight based on its total protein content, preferentially between 5% and 8% by weight.

The total protein content may be determined by any protocol well known to those skilled in the art, for instance assay of the total amount of amino acids. Preferentially, the total nitrogen will be assayed according to the Dumas method and the value will be multiplied by a coefficient of 6.25. The total protein content is between 10% and 30%, preferentially between 15% and 25%.

Lysine (IUPAC-IUBMB abbreviations: Lys and K) is an α-amino acid, the L enantiomer of which is one of the 22 proteinogenous amino acids. Its structural formula is as follows:

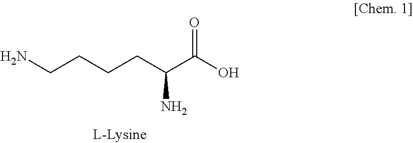

By virtue of its two amine functions, lysine, when placed in contact with a reducing sugar and when they are heated, reacts strongly by participating in the “Maillard” reactions.

The reaction Maillard is a chemical reaction which corresponds to the action of reducing sugars on proteins, and contributing notably to the appearance of brownish colourings and of odorous compounds (through the generation of aldehydes and ketones).

The proteins of leguminous plants and in particular of pea, are very rich in lysine. It is taught, in particular in “Les protéines de pois: de leur fonction dans la graine à leur utilisation en alimentation animale [Pea proteins: from their function in the seed to their use in animal feed]” from C. Perrot, published in 1995, that “heating of (pea) proteins in the presence of reducing sugars (fructose, lactose, etc.) results in the formation of numerous complex polymers, notably involving lysine. This reaction, called non-enzymatic browning or Maillard reaction, also contributes towards lowering the digestibility of proteins. Finally, heating may also induce racemization of the amino acids, i.e. passage from their natural L form to the D form, which is no longer recognized by the digestive enzymes (Zagon et al. 1994).”. It is also taught that “(pea) albumins have a higher content of sulfur amino acids and of lysine.”. However, the pea soluble fraction is the albumin-rich fraction. Those skilled in the art would have thus considered that mixing, followed by drying, of the pea internal fibres containing sugars and the albumin-rich, and therefore lysine-rich, pea soluble fraction, would have been disastrous as it would be bound to generate a product for which the digestibility of the organic matter, including that of lysine, would have been strongly reduced.

Finally, even more preferentially, the product according to the invention is characterized in that the total digestibility of its organic matter for monogastric animals is greater than 75%.

In the present application, the term “organic matter” means the total amount of dry matter after subtraction of the ash, which consists mainly of inorganic salts.

In the present application, the term “monogastric animal” means any domesticated animal (pig, poultry) having only one gastric pouch, as opposed to ruminants, which have four. Monogastric animals have particular difficulty in digesting foods that have undergone Maillard reactions.

The protocol used for determining the total digestibility of the organic matter, known as the CVB Protocol of 2005, is well known to those skilled in the art.

A second subject of the invention relates a process for producing the product derived from the mixing of soluble fractions from leguminous plants and of pulps from leguminous plants, the solids content of which is greater than 85% by weight, preferentially greater than 90% by weight, even more preferentially greater than 94% by weight, comprising the following steps:

i) pre-treatment of leguminous plant seeds;
ii) wet separation of the constituents of the leguminous plant seeds into four fractions: a starch fraction, a pulp fraction, a protein fraction of globulin type and a soluble fraction;
iii) mixing of the pulp fraction and the soluble fraction separated in the preceding step ii);
iv) drying of the mixture obtained in step iii).

Preferentially, the leguminous plant is chosen from the list composed of faba bean and pea. Pea is particularly preferred.

The first step of pre-treatment of pea seeds consists in preparing for the next steps. The external fibres are separated from the actual seeds. The seeds may then undergo steps of cleaning, sieving (e.g. for separating seeds and stones), soaking, bleaching, toasting. Preferably, if a bleaching step is performed, the heat treatment protocol will be of 3 min at 80° C.

The second step is described in detail in patent application EP1400537, which is incorporated herein by reference. The principle of this process consists in grinding the leguminous plant seed to obtain a meal, which is then resuspended in water. Using decanters and hydrocyclones, the starch and the internal fibres (pulps) are separated. The residual solution, having a high protein content, is acidified to a pH of about 4.5, and then undergoes a heating step to coagulate the proteins of globulin type, which will be separated by centrifugation. The residual solution consists of the “soluble fraction”.

Any other wet extraction process which results in the four fractions: a starch fraction, a pulp fraction, a protein fraction of globulin type and a soluble fraction, is however also conceivable. It is also possible to obtain a concentrate via a dry process (turbo-separation or air-classification) and then to continue extracting the various fractions using a wet process.

The third step consists of intimate mixing of the pulp fraction pulp and the soluble fraction. It is to the patent proprietor's credit to have shown that if this mixing is not properly performed, the running of the drier deteriorates and the final qualities of the product after drying degrade rapidly.

Preferably, the ratio, expressed in solids content, of the soluble fraction to the pulp fraction, is between 0.8/1.2 and 1.2/0.8, preferentially between 1/1 and 1.2/0.8, preferentially 1/1.

Preferably, the mixing of the pulp fraction and the soluble fraction is performed in a high-performance mixer for a residence time of less than 5 min.

Preferably, a mixer of Ploughshare® type from Lodige is used. The aim is here to obtain a homogeneous intimate mixture of the two fractions. Such a result makes it possible to then obtain optimal drying, without any sticking in the equipment.

Even more preferably, the soluble fraction is pre-concentrated to between 30% and 50% by weight, preferentially to a solids content (S.C.) of 50% by weight, before being mixed with the fibres, also known as the pulp fraction. The soluble fraction thus concentrated has a content of raw protein (N*6.25) of between 20% and 40% by weight relative to the solids.

The fourth step consists in drying the mixture thus obtained.

Preferably, a ring-dryer technology will be used, preferably with recycling of the evaporation mists.

This technology is an improvement of the drier technology of flash type (flash-dryer), where the wet mixture is dispersed into a heated flow of air (or of gas) which conveys it through a drying conduit. By using the heat of the air flow, the material dries as it is conveyed. The product is separated using cyclones and/or bag filters.

For even higher thermal efficiency, recycling of the exhaust gases may be used. This configuration of partial gas recycling (PGR) is particularly preferred in the case of the product according to the invention.

The ring dryer differs from a flash type dryer (flash dryer) in that it incorporates a classifier (collector), which enables the recirculation of a partial amount of the semi-dried product into the initial heating area for further drying and dispersion.

The air outlet temperature will be managed in order to be between 80° C. and 130° C., preferentially between 90° C. and 120° C., even more preferentially between 100° C. and 110° C. The air inlet temperature will be between 180° C. and 300° C., preferentially between 240° C. and 265° C.

The solids content at the inlet must be greater than 65% by weight, preferentially greater than 70% by weight. Specifically, if the solids content is lower than this threshold, the Applicant has shown that the drying will be less successful, resulting in problems of stickiness in the drying equipment. Moreover, these conditions make it possible to obtain a final product with a mean diameter of 1000 microns.

Finally, the invention also relates to the industrial use of the mixture of pea soluble fractions and of pea pulp fractions, the solids content of which is greater than 90% by weight, preferentially greater than 92% by weight, even more preferentially greater than 94% by weight, in industrial applications such as food for human consumption, animal feed, pharmaceuticals or cosmetics.

A lysine-rich nutrient, having a guaranteed content, and also a high content of digestible fibres, which is not strongly coloured and which is stable towards subsequent heat treatments, can then be made available to the animal feed industry.

It is also conceivable to provide a product with more colour, the lysine content of which being still guaranteed. Specifically, as shown in the Examples section, the product obtained according to the invention, after controlled heating, makes it possible to generate colouring, but with limited loss of lysine. This behaviour is entirely unique compared with the other sectors of corn or wheat type.

The invention will be better understood with the aid of the examples below, which however will not limit its scope.

### DESCRIPTION OF THE FIGURES

FIG. 1 shows the evolution with time of the L*a*b* colouring of the wheat-based product of the prior art. The x-axis corresponds to time, expressed in minutes, and the y-axis corresponds to the L*a*b* colouring.

FIG. 2 shows the evolution with time of the L*a*b* colouring of the pea-based product according to the invention. The x-axis corresponds to time, expressed in minutes, and the y-axis corresponds to the L*a*b* colouring.

FIG. 3 shows the evolution with time of the levels of reducing sugars and of lysine of the wheat-based product of the prior art. The x-axis corresponds to time, expressed in minutes, the y-axis (left) corresponds to the level of reducing sugars expressed as a weight percentage relative to the gross weight and the y-axis (right) corresponds to the level of lysine expressed as a weight percentage relative to the gross weight.

FIG. 4 shows the evolution with time of the levels of reducing sugars and of lysine of the pea-based product according to the invention. The x-axis corresponds to time, expressed in minutes, the y-axis (left) corresponds to the level of reducing sugars expressed as a weight percentage relative to the gross weight and the y-axis (right) corresponds to the level of lysine expressed as a weight percentage relative to the gross weight.

### EXAMPLES

### Example 1: Production of a Product According to the Invention

After dehulling the external fibres using a hammer mill, the pea seeds are milled to produce a meal. 300 kg of meal with a solids content of 87% are then soaked in water at a final concentration of 25% on a dry weight basis, at a pH of 6.5 for 30 minutes at room temperature. 1044 kg of meal suspension containing 25% by weight of solids (thus 261 kg of dry meal) are then introduced with 500 kg of water into a hydrocyclone array adapted from an industrial potato starch processing unit. This separation leads to the production of a light phase consisting of the mixture of protein, internal fibres (pulp) and soluble matter. The heavy phase, containing the starch, is discarded.

The light phase at the hydrocyclone outlet contains as a mixture (142 kg of solids in total): fibres (about 14.8% by weight, i.e. 21 kg of solids), protein (about 42.8% by weight, i.e. 60.8 kg of solids) and soluble matter (about 42.4% by weight, i.e. 60.2 kg of solids). It is then brought to a solids content of 11.4%. The fibres are separated out on centrifugal decanters of Westfalia type used in an industrial potato starch processing unit. The light phase at the outlet of the centrifugal decanter contains a mixture of protein and of soluble matter, while the heavy phase contains the pea fibres. The heavy phase contains 105 kg of fibres with a solids content of 20%. It is noted that virtually all of the fibres are indeed found in this fraction. This fraction will be referred to hereinbelow as the “internal pea fibres” and corresponds to the pulp fraction.

As for the light fraction, it contains 1142 kg of a dissolved mixture of soluble matter and protein. The proteins were coagulated at their isoelectric point by adjusting the light phase at the outlet of the centrifugal decanter to a pH of 4.6 and heating this solution at 70° C. for 20 min. After precipitation of the proteins, the sediment, containing 56 kg of proteins (86% of N*6.25 on a dry basis) at 93% by weight of solids, was discarded. The liquid fraction, which will be called “pea soluble fraction” was concentrated by vacuum evaporation to about 50% by weight of SC.

The two fractions “pea internal fibres” and “pea residual soluble fraction” were mixed using a Lodige FM130 Paddle mixer, with a respective ratio of 45/55 on a solids basis. The solids content was adjusted to about 70% by weight. The mixture was dried using a DEDERT brand ring-dryer. The evaporation capacity was 60-80 kg of water/h. The ring-dryer was configured in “PGR” mode, i.e. with recycling of the evaporation mists. The running of the ring-dryer was adjusted in order to guarantee a temperature of the air inlet of 250° C. and of air outlet of 115° C.

### Example 2: Temperature Stability of the Invention Versus Products from the Prior Art

The aim of Example 2 was to demonstrate the impact of the drying conditions on the properties of the product according to the invention, when it is exposed to temperature.

For comparative purposes, use was made of reference by-products from the wheat sector, which were mixed in a certain ratio and dried under similar conditions (40° C., 200 mbar, 68 h).

The analyses of the two products obtained were as follows:

TABLE 1

| | Pea-based | Wheat- |
|---|---|---|
| | product | based |
| | according | product |
| | to the | of the |
| | invention | prior art |
| SC (% by weight) | 90.8 | 89.8 |
| Aw or water | 0.40 | 0.53 |
| activity (20° C.) | | |
| Lysine content | 1.04 | 0.48 |
| (as % by gross weight) | | |
| Reducing sugars | 3.70 | 5.10 |
| (as % by gross weight) | | |

Samples containing 100 g of the two samples were then introduced into hermetically sealed aluminium bags, and then introduced into an oven heated to 100° C. Samples were collected on a regular basis over 120 min, at regular intervals.

The following were analysed on these samples:
Visual observation of the colour (DDGS colour scale);
Measurement of the L*a*b* colouring by spectrophotometry;
Analysis of the lysine content.

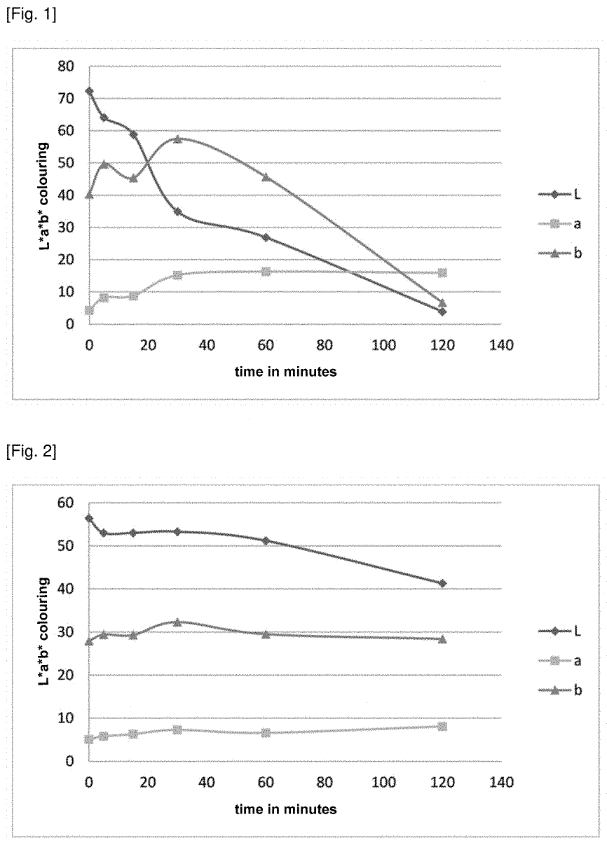

The L*a*b* colourings described in [FIG. 1] and [FIG. 2] did indeed provide evidence for this observation.

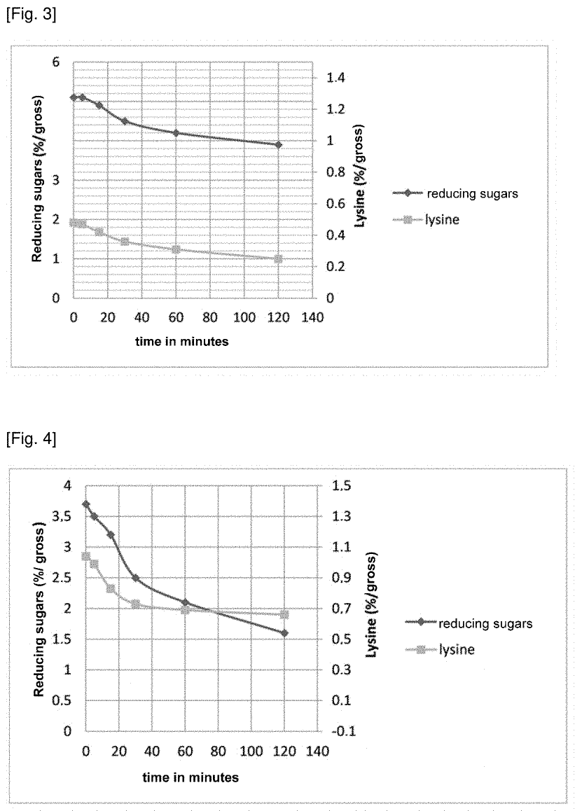

The analysis of the lysine in the collected samples gave the results described in [FIG. 3] and [FIG. 4].

Surprisingly, it is noted that:
The appearance of colouring was greater with the wheat-based product according to the prior art;
The loss of lysine was ultimately only 33% by weight in the product according to the invention, whereas the loss was 50% by weight in the wheat-based product from the prior art;
This result was the opposite of the previous results regarding colour development. Those skilled in the art would have expected greater degradation of lysine in the product according to the invention.

These results make it possible to confirm that the product according to the invention shows very special behaviour, and that, if the process performed by the Applicant is followed, a product with a guaranteed colouring and lysine content and which corresponds to the expectations of the animal nutrition sector, is obtained.

### Example 3: Assessment of the Nutritional Properties of the Product According to the Invention for Feeding Pigs

The digestibility of the product obtained according to the invention in Example 1 was studied in vivo.

This study compared the total digestibility of a control basic ration A, of a ration B with the incorporation of 25% by weight of yellow peas (Canadian origin), and of two rations C and D containing 15% and 30% by weight respectively of the product obtained according to the invention in Example 1. The rations were presented as granules 3 mm in diameter, with a suitable methionine supplementation, in order to compensate for the usually low content of methionine in pea products.

The pigs were fed twice a day, up to a feeding level equivalent to 3.2 times the maintenance requirement. The average live weight of the pigs at the start of the experiment was 49.5 kg.

This study showed digestibility values of the organic matter which were in accordance with the values expected for the yellow pea tested (88.7%), in comparison with the value in the reference tables for pig feed (INRA 2002=90%, CVB 2006=92%). This validated the relevance of the retained experimental protocol.

The average digestibility of the organic matter for the product obtained according to the invention in Example 1 was found to be 82%.

This result is entirely surprising: the difference in digestibility between the dried by-product and the original raw material was less than 10 units, while it is usual to find a discrepancy of greater than 20 units according to the tables, when the digestibility of the organic matter in pigs is compared between the original raw material and the by-product composed of co-dried fibres and soluble matter. According to the previously mentioned INRA study, the European wheat stillage grain (Wheat DDGS) show an average digestibility of 68%, compared with 90% for wheat. For the corn stillage grain (Corn DDGS), the values are 69% as opposed to 91% for corn.

1. Product derived from the mixing of a soluble fraction from leguminous plants and of pulps from leguminous plants, wherein its solids content is greater than 85% by weight, preferentially greater than 90% by weight, even more preferentially greater than 94% by weight.

2. Product according to claim 1, wherein the leguminous plant is preferentially chosen from the list consisting of faba bean and pea.

3. Product according to claim 1, wherein 90% of the product constituents have a size of between 50 microns and 3000 microns, preferentially between 300 microns and 1000 microns.

4. Product according to claim 1, wherein its colouring according to the “L*a*b*” technique, wherein its component L is greater than 30, preferentially greater than 40, even more preferentially greater than 50.

5. Product according to claim 4, wherein its component a is less than 20, preferentially less than 10, and its component b is greater than 25, preferably greater than 30, preferably greater than 40, preferentially greater than 50.

6. Product according to claim 1, wherein its lysine content is between 3% and 10% by weight of its total protein content, preferentially between 5% and 8%.

7. Product according to claim 1, wherein the total digestibility of its organic matter for monogastric animals is greater than 75%.

8. Process for preparing the product according to claim 1, comprising the following steps:
i) pre-treatment of leguminous plant seeds;
ii) wet separation of the constituents of the leguminous plant seeds into four fractions: a starch fraction, a pulp fraction, a protein fraction of globulin type and a soluble fraction;
iii) mixing of the pulp fraction and the soluble fraction separated in the preceding step ii); and
iv) drying of the mixture obtained in step iii).

9. Process for preparing the product according to claim 8, wherein the ratio, expressed in solids content, of the soluble fraction to the pulp fraction is between 0.8/1.2 and 1.2/0.8, preferentially 1/1.

10. Process for preparing the product according to claim 8, wherein the soluble fraction is pre-concentrated to between 30% and 50% by weight, preferentially to 50% by weight of solids before being mixed with the pulp fraction.

11. Process for preparing the product according to claim 8, wherein the mixing of the pulp fraction and of the soluble fraction is performed in a high-performance mixer for a residence time of less than 5 min.

12. Process for preparing the product according to claim 8, wherein drying is performed via a ring-dryer technology, preferably with recycling of the evaporation mists.

13. Industrial use of the product according to claim 1 in industrial applications such as food for human consumption, animal feed, pharmaceuticals, or cosmetics.
